(19) United States
(12) Patent Application Publication
(10) Pub. No.: US 2004/0106771 A1
(43) Pub. Date: Jun. 3, 2004

(54) Natural antibodie active against hiv virus

(76) Inventor: Radmila Metlas, Guernsey (GB)

Correspondence Address: YOUNG & THOMPSON, 745 SOUTH 23RD STREET 2ND FLOOR, ARLINGTON, VA, 22202

(21) Appl. No.: 10/469,870

(22) PCT Filed: Mar. 6, 2002
(86) PCT No.: PCT/EP02/02454

(30) Foreign Application Priority Data
Mar. 9, 2001 (IT) MI2001A000500
Oct. 31, 2001 (IT) MI2001A002285

(51) Int. Cl. A61K 39/395; A61K 39/12; C07K 2/00; C07K 4/00; C07K 5/00; C07K 7/00; C07K 14/00; C07K 16/00; C07K 17/00; A61K 38/00

(52) U.S. Cl. 530/300; 424/130.1; 424/204.1

(57) ABSTRACT

A preparation of human antibodies capable of neutralizing HIV-1 virus and the use thereof in the treatment of HIV infection.

The present invention relates to antibodies neutralizing the Human Immunodeficiency Virus. Said antibodies are present in sera from normal subjects and can be used to prevent the HIV infection or to delay the onset of illness in serum positive patients. The present invention further relates to pharmaceutical preparations containing said antibodies and the use thereof in passive immunotherapy of HIV infection.

### BACKGROUND OF THE INVENTION

The induction of effective immune response to HIV-1 has often been restricted by the inability of any vaccine candidate to elicit antibodies capable of neutralizing infectivity of primary HIV isolates from infected individuals (1,2). On the other hand, from the analysis of sera from infected subjects resulted that neutralization of primary virus tends to be weak and sporadic (3,4). However, albeit it is difficult to raise antibodies, either during the infection or with experimental vaccines, monoclonal antibodies exist, such as IgG1b12, 2F5 and 2G12 (5-7), which are capable of neutralizing primary viruses of subtypes A-E. Said antibodies derive from subtype B—infected individuals and they are not raised by vaccination (8).

The earliest detectable HIV-1 specific antibody is of the IgG isotype (12), suggesting a nonconventional primary response (13). Furthermore, there are findings indicating that HIV-1 antigens react with pre-existing, antibody repertoires (14). Cross-reactive antibodies that recognize the HIV-1 envelope have been reported (15).

It was suggested that a strong early humoral response in HIV-1 infection may not be beneficial (16) as it would result in monoclonal and polyclonal antibody population instead of normal polyclonal response (13). It has been suggested that anti-idiotypic antibody 1F7 may broaden immune response to HIV antigens (17).

In our earlier studies, the homology of HIV envelope protein with IgG variable protein was predicted (18,19). However, recent data suggest that HIV-1 gp120 immunodominant epitope and a fraction of naturally-occurring IgG antibodies share complementary structure (20.21).

### DISCLOSURE OF THE INVENTION

It has now been found that a group of antibodies present in sera from normal, not HIV—infected individuals, are capable of neutralizing HIV virus.

The antibody active fraction was purified from sera of normal subjects by affinity chromatography using a Sepharose resin to which total human IgG had been coupled. Observation evidenced that such anti-IgG antibodies fraction is capable of neutralizing HIV-1 infection in PBMC. The experiments were repeated using different anti-IgG preparations of various concentrations, obtained from commercial serum specimens, using affinity purified total IgG antibodies as negative control and HIVIG and mAb 4117C antibodies as positive control. The latter proved to be inactive to 92HT593B. Neutralization curves shown in FIG. 1 relate to a representative experiment carried out with HIV-1 primary isolates 92HT593B and SF162WT as well as with NLHX-ADA recombinant virus. Anti-IgG antibodies proved to be active against 92HT593B and NLHX-ADA at a 50% inhibiting concentration ranging from less than 1 to 7 μg/ml.

There is little possibility for the neutralizing effects observed with the preparation of anti-IgG antibodies to be due to co-purified chemokines from normal sera, as the concentration of these substances does not exceed 20 ng per ml of serum and no specific bands were revealed by PAGE. Furthermore, molecules below 50 kDa were removed by dialysis of the antibodies preparations. PAGE analysis of anti-IgG preparations revealed predominant presence of IgG and very small traces of contaminant proteins.

A possible explanation of the observed effects is that HIV-1 antigenic determinants share complementary structure with the variable regions of natural antibodies belonging to the immune network (9,23), but in no way this or other explanations limit the invention.

Therefore, a first aspect of the invention relates to a preparation of human antibodies raised against the total human IgG fraction, capable of neutralizing HIV-1. Said preparation can be obtained subjecting sera from normal, not HIV-infected subjects, to affinity chromatography using resins coupled to total human IgG fraction. The final concentration of isolated anti-IgG antibodies will be preferably comprised within the range of 0.1-1000 μg/ml, more preferably within the range of 0.11-100 μg/ml. The starting material consists of a pool of sera from normal subjects. According to a further aspect, the invention relates to the use of the preparation of the invention in the prophylactic or therapeutical treatment of HIV infection, preferably HIV-1 infection.

For the use in therapy, the preparation of the invention will be suitably formulated with pharmaceutically acceptable excipients. The latter include buffering agents, stabilizing agents, solubilizers, diluents, isotonicity agents and the like. The preparation will preferably be administered parenterally, preferably through the intravenous, intradermal or intramuscular route. According to a preferred embodiment, the preparation will be used in passive immunotherapy of HIV-1 infection.

### DISCLOSURE OF THE FIGURES

FIG. 1

Neutralization of primary HIV-1 isolates SF162WT and 92HT593B and recombinant NL-HX-ADA virus by anti-IgG antibodies. Each titration curve represents data from a single experiment. Concentrations of anti-IgG in the various experiments were respectively 109.7, 12.3 and 80.0 μg/ml, for total IgG between 1000 and 3333 μg/ml and for HIVIG (10000 mg/ml). Anti-IgG and total IgG preparations were obtained as described in the following. HIVIG was prepared from sera of HIV-1 infected subjects.

FIG. 2

SDS-PAGE separation of affinity purified antibodies. Anti-IgG and total IgG antibodies were prepared as described below. Electrophoretic separation of anti-IgG antibodies revealed a prevalent content of IgG. In addition, although weak, bands of high molecular weight proteins were also present.

### EXAMPLE 1

### Preparation of Anti-IgG Antibodies

Anti-IgG antibodies were prepared from a pool of two normal human sera by affinity chromatography using Sepharose beads (CNBr-activated Sepharose 4B) to which total human IgG (purified on GammaBind Sepharose 4B affinity column) had previously been coupled. In principle, 5-7 mg of IgG antibodies/0.5-0.6 g of Sepharose beads were used. Binding was carried out following the indications of the manufacturer. The column was equilibrated with 5×PBS and heat-inactivated serum (1 ml) was loaded and diluted to 1:1 by 5×PBS. Incubation was carried out for 1 hour at room temperature or overnight at 4° C. After washing with 30 ml of 5×PBS, bound antibodies were eluted using 0.1 M citrate, pH=2.5 into test-tubes containing base-TRIS. The collected eluate was concentrated, dialyzed (Centricon YM, 30000 MW cut-off) and analyzed through 10% SDS-PAGE, predominantly revealing the IgG band (FIG. 2).

### EXAMPLE 2

### Neutralization of HIV-1 Isolates by Anti-IgG Antibodies

Virus neutralization was evaluated in PBMC following a previously published protocol (22). Neutralization assays were carried out with four different preparations of antibodies in five separated experiments.

Table 1 summarizes neutralization data against two HIV primary isolates (92HT593B, SF162WT) and a recombinant NL-HX-ADA virus.

HIVIG and mAb4117C were used as positive controls; Protein-G IgG was used as negative control.

In this test, all of the preparations of anti-IgG antibodies proved capable of inhibiting PBMC infection by 92HT593B and NL-HX-ADA, whereas no neutralizing activity against SF162WT was observed.

TABLE
Viral neutralization titers of purified Ig from sera from
HIV-1 infected (a) and normal (b) subjects

| | Recombinant |
|---|---|
| Primary isolate | virus |

| | | | |
|---|---|---|---|
| Antibodies Preparat. | 92HT593B | SF162WT | NLHX-ADA |

| Assay | μg/ml | % neut | μg/ml | % neut | μg/ml | % neut. |
|---|---|---|---|---|---|---|
| Negative control: | | | | | | |
| Protein G IgG b) | 150 | 8.3 | 150 | 2.9 | 150 | −6.8 |
| | 50 | 1.1 | 50 | −0.5 | 50 | −4.4 |
| | 16.7 | 2.2 | 16.7 | 2.7 | 16.7 | −4.4 |
| Positive control: | | | | | | |
| mAb4117C a) | 100 | 16.1 | 100 | 100 | 100 | 98.7 |
| | 33.3 | 9.0 | 33.3 | 100 | 33.3 | 96.0 |
| | 11.1 | 5.2 | 11.1 | 100 | 1.1 | 55.0 |
| | 3.7 | 0.4 | 3.7 | 80.4 | 3.7 | 23.2 |
| | 1.2 | −0.6 | 1.2 | 57.4 | 1.2 | −8.6 |
| Positivc control: | | | | | | |
| HIVIG a) | 100 | 88.0 | 100 | 57.4 | 100 | 93.4 |
| | 33.3 | 62.0 | 33.3 | 39.9 | 33.3 | 94.8 |
| | 11.1 | 44.4 | 11.1 | 2.1 | 11.1 | 92.4 |
| | 3.7 | 39.4 | 3.7 | −14.4 | 3.7 | 87.2 |
| | 1.23 | 22.2 | 1.23 | 0.7 | 1.23 | 69.2 |
| | 0.41 | 8.3 | 0.41 | −3.7 | 0.41 | 54.5 |

| Anti-IgG b) | 1 | 1 | 1.87 | 57.4 | NA | ND | |
|---|---|---|---|---|---|---|---|
| | | | 0.62 | 37.6 | | | |
| | | | 0.21 | 6.1 | | | |
| Anti-IgG b) | 1 | 2 | 1.23 | 71.8 | NA | ND | |
| | | | 0.41 | 48.9 | | | |
| | | | 0.14 | 14.4 | | | |
| Anti-IgG b) | 2 | 1 | 8.00 | 42.0 | NA | 8.00 | 60.3 |
| | | | 2.67 | 24.1 | | 2.67 | 14.6 |
| | | | 0.89 | 5.7 | | 0.89 | 1.3 |
| Anti-IgG b) | 3 | 1 | 2.17 | 46.4 | NA | ND | |
| | | | 0.72 | 27.1 | | | |
| | | | 0.24 | 3.3 | | | |
| Anti-IgG b) | 4 | 1 | 10.97 | 39.8 | NA | 10.97 | 68.7 |
| | | | 3.66 | — | | 3.66 | 54.0 |
| | | | 1.22 | — | | 1.22 | 45.0 |

Literature

1. Mascola, J R, Snyder, S W, Weislow, O S. Belay, S M, Belshe, R B, Schwartz, D H, Clements, M L, Dolin, R, Graham, B S, Gorse, G J, Keefer, M C, McElrath, M J, Walker, M C, Wagner, K F, McNeil, J G, McCutchan, F E, and Burke, D S: Immunization with envelope subunit vaccine products elicits neutralizing antibodies against laboratory-adapted but not primary isolates of human immunodeficiency virus type 1. J Infect Dis 1996; 173:340-348.

2. Montefiori, D C, and Evans, T G: Toward and HIV type 1 vaccine that generates potent, broadly cross-reactive neutralizing antibodies. AIDS Res Hum-Retroviruses 1999;15:689-698;

3. Moore, J P, Coa, Y, Leu, Qin, L, Korber, B, and Ho, D D: Inter and intraclade neutralization of human immunodeficiency virus type 1: genetic clades do not correspond to neutralization serotypes but partially correspond to gp120 antigenic serotypes. J Virol 1996;70:427-444;

4. Weber, J, Fenyo, E M, Beddows, S, Kaleeby, P, Bjorndal, A, and the WHO Network for HIV Isolation and Characterization: Neutralization serotypes of HIV-1 field isolates are not predicted by genetic subtype. J Virol 1996;70:7827-7832;

5. Burton, D R, Pyati, J, Koduri, R, Sharp, S J, Thornton, G B, Parren, P W H I, Sawyer, L S W, Hendry, R M, Dunlop, N, Nara, P L, Lamacchia, M, Garratty, E, Stiehm, E R, Bryson, Y J, Cao, Y, Moore, J O, Ho, D D, and Barbas III, C F: Efficient neutralization of primary isolates of HIV-1 by a recombinant human monoclonal antibody. Science 1994;266:1024-1027;

6. Mascola, J R, Louder, M K, VanCott, T C, Sapan, C V, Lambert, J S, Muenz, L R, Bunow, B, Birx, D L, and Robb, M L: Potent and synergistic neutralization of human immunodeficiency virus (HIV) type 1 primary isolates by hyperimmune anti-HIV immunoglobulin combined with monoclonal antibodies 2F5 and 2G12. J Virol 1997;71:7198-7206;

7. Trkola, A, Pomales, A B, Yuan, H, Kober, B, Maddon, P J, Allaway, G P, Katinger, H, Barbas III, C F, Burton, D R, Ho, D D, and Moore, J P: Cross-clade neutralization of primary isolates of human immunodeficiency virus type 1 by human monoclonal antibodies and tetrameric CD4-IgG. J Virol 1995;69:6609-6617;

8. Moore, J P, and Trkola, A: JIV type 1 coreceptors, neutralization serotypes, and vaccine development. AIDS Res Human Retroviruses 1997;13:733-736;

9. Coutinho A: Beyond clonal selection and network. Immunological Reviews 1989;110:63-67;

10. Avrames, S: Natural autoantibodies: from “horror autotexicus” to “gnothi seauton”. Immunol Today 1991;12:154-159;

11. Moller, G: Effect of anti-immunoglobulin sera on B lymphocyte functions. Scand J Immunol 1978;8:469-474;

12. Race, E M, Ramsey, K M, Lucia, H L, and Cloyd, M W: Human immunodeficiency virus elicits antibody not detected by standard tests; implications for diagnostics and viral immunology. Virology 1991;184:716-722;

13. Nara, P L, and Garrity, R: Deceptive imprinting: a cosmopolitan strategy for complicating vaccination. Vaccine 1998;16:1780-17787;

14. Zubler, R H, Perrin, L H, Doucet, A, Zhang, X, Huang, Y P, and Miescher, P A: Frequencies of HIV-reactive B cells in seropositive and seronegative individuals. Clin Exp Immunol 1991; 87:31-36;

15. Davis, D, Chaudhri, B, Stephens, D M, Carne, C A, Willers, C, and Lachmann, P J: The immunodominance of epitopes within the transmembrane protein (gp41) of human immunodeficiency virus type 1 may be determined by the host's previous exposure to similar epitopes on unrelated antigens. J Gen Virol 1990;71:1975-1983;

16. Velijovic, V, Metlas, R, Kohler, H, Urnovitz, H B, Prljic, J, Velikovic, N, Johnson, E, and Muller, S: AIDS epidemic at the beginning of the third millennium: time for a new AIDS vaccine strategy. Vaccine 2000;19:1855-1863;

17. Wang, Q L, Wang, H T, Blalock, E, Moller, S, and K″hlerm H: Identification of an idiotypic peptide recognized by autoantibodies in immunodeficiency virus-1-infected individuals. Idiotype-specific autoantibodies in AIDS 1995;96:775-780;

18. Veljkovic, V, and Metlas, R: Identification of immunoglobulin recombination elements in HIV-1 envelope gene. Immunol Lett 1991;31:11-14;

19. Metlas, R, Veljkovic, V, Paladini, D R, and Pongor, S: Protein and DNA sequence homology between the V3 loop of human immunodeficiency virus type 1 envelope protein gp120 and immunoglobulin variable region. Biochem Biophys Res Commun 1991;179:1056-1062;

20. Metlas, R. Skerl, V, Veljkovic, V, Colombatti, A, and Pongor, S: Immunoglobulin-like domain of HIV-1 envelope glycoprotein gp120 encodes putative internal image of some common human proteins. Viral Immunol 1994;7:215-219;

21. Metlas, R, Trajkovic, D, Srdic, T, Veljkovic, V, and Colombatti, A: Anti-V3 and anti-IgG antibodies of normal individuals share complementariry structures. JAIDS 1999;21:266-270;

22. Pinter, A, Honnen, W J, Tilley, S A: Conformational changes affecting the V3 and CD4-binding domains of human immunodeficiency virus type 1 gp120 associated with env processing and with binding of ligands to these sites. J Virol 1993;67:5692-5697;

23. Holmberg, D, Andersson, A, Carlsson, L, and Forsgren, S: Establishment and functional implications of B-cell connectivity. Immunological Reviews 1989; 110:89-103;

24. Ochsenbein, A F, and Zinkernagel, R M: Natural antibodies and complement link innate and acquired immunity. Immunol Today 2000;21:624-629;

25. Fearon, D T, and Locksley, R M: The instructive role of innate immunity in the acquired immune response. Science 1996;272:50-54.

1. Human antibodies aganist human IgG total fraction, capable of neutralizing HIV-1.

2. Antibodies as claimed in claim 1, obtainable by subjecting sera from normal, non HIV-infected subjects, to affinity chromatography using resins bound to human IgG total fraction.

3. Pharmaceutical preparation containing the antibodies of claims 1-2.

4. The use of antibodies of claims 1 and 2 for the preparation of a medicament for use in the prevention or in the treatment of HIV infections.

5. The use of antibodies of claims 1 and 2 for the preparation of a medicament for use in passive immunotherapy of HIV-1 infection.
